(19) United States
(12) Patent Application Publication
(10) Pub. No.: US 2014/0030748 A1
(43) Pub. Date: Jan. 30, 2014

(54) METHOD AND SYSTEM TO MANAGE DIABETES USING MULTIPLE RISK INDICATORS FOR A PERSON WITH DIABETES

(71) Applicant: Thomas SCHAIBLE, Phoenixville, PA (US)

(72) Inventor: Thomas SCHAIBLE, Phoenixville, PA (US)

(73) Assignee: LifeScan, Inc., Milpitas, CA (US)

(21) Appl. No.: 13/560,627

(22) Filed: Jul. 27, 2012

(51) Int. Cl. G01N 27/00; G06F 19/00; C12M 1/40

(52) U.S. Cl. 435/14; 435/287.1; 702/19

(57) ABSTRACT

Described are methods and systems to annunciate to the patient of the components involved in each of the daily risk range based on the glucose measurements to assist the patient in identification of whether it is hypoglycemia or hyperglycemia are driving the daily risk range of the measured glucose values.

### BACKGROUND

Diabetes mellitus is a chronic metabolic disorder caused by an inability of the pancreas to produce sufficient amounts of the hormone drug so that the metabolism is unable to provide for the proper absorption of sugar and starch. This failure leads to hyperglycemia, i.e. the presence of an excessive amount of analyte within the blood plasma. Persistent hyperglycemia has been associated with a variety of serious symptoms and life threatening long term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to always maintain the level of blood analyte within normal limits. Such glycemic control is achieved by regularly supplying external drug to the body of the patient to thereby reduce the elevated levels of blood analyte.

External drug was commonly administered by means of multiple, daily injections of a mixture of rapid and intermediate acting drug via a hypodermic syringe. While this treatment does not require the frequent estimation of blood analyte, it has been found that the degree of glycemic control achievable in this way is suboptimal because the delivery is unlike physiological drug production, according to which drug enters the bloodstream at a lower rate and over a more extended period of time. Improved glycemic control may be achieved by the so-called intensive drug therapy which is based on multiple daily injections, including one or two injections per day of long acting drug for providing basal drug and additional injections of rapidly acting drug before each meal in an amount proportional to the size of the meal. Although traditional syringes have at least partly been replaced by drug pens, the frequent injections are nevertheless very inconvenient for the patient, particularly those who are incapable of reliably self-administering injections.

Substantial improvements in diabetes therapy have been achieved by the development of the drug delivery device, relieving the patient of the need for syringes or drug pens and the administration of multiple, daily injections. The drug delivery device allows for the delivery of drug in a manner that bears greater similarity to the naturally occurring physiological processes and can be controlled to follow standard or individually modified protocols to give the patient better glycemic control.

In addition, delivery directly into the intraperitoneal space or intravenously can be achieved by drug delivery devices. Drug delivery devices can be constructed as an implantable device for subcutaneous arrangement or can be constructed as an external device with an infusion set for subcutaneous infusion to the patient via the transcutaneous insertion of a catheter, cannula or a transdermal drug transport such as through a patch. External drug delivery devices are mounted on clothing, hidden beneath or inside clothing, or mounted on the body and are generally controlled via a user interface built-in to the device or on a separate remote device.

Drug delivery devices have been utilized to assist in the management of diabetes by infusing drug or a suitable biologically effective material into the diabetic patient at a basal rate with additional drug or “bolus” to account for meals or high analyte values, levels or concentrations. The drug delivery device is connected to an infuser, better known as an infusion set by a flexible hose. The infuser typically has a subcutaneous cannula, adhesive backed mount on which the cannula is attached thereto. The cannula may include a quick disconnect to allow the cannula and mount to remain in place on the skin surface of the user while the flexible tubing is disconnected from the infuser. Regardless of the type of drug delivery device, blood analyte monitoring is required to achieve acceptable glycemic control. For example, delivery of suitable amounts of drug by the drug delivery device requires that the patient frequently determines his or her blood analyte level and manually input this value into a user interface for the external pumps, which then calculates a suitable modification to the default or currently in-use drug delivery protocol, i.e. dosage and timing, and subsequently communicates with the drug delivery device to adjust its operation accordingly. The determination of blood analyte concentration is typically performed by means of an episodic measuring device such as a hand-held electronic meter which receives blood samples via enzyme-based test strips and calculates the blood analyte value based on the enzymatic reaction.

In recent years, continuous analyte monitoring has also been utilized with drug delivery devices to allow for greater control of the drug(s) being infused into the diabetic patients. In addition to glucose monitoring, people with diabetes often have to perform drug therapy such as, for example, insulin dosing. People with diabetes may self-administer insulin to reduce their blood glucose concentration. There are a number of mechanical devices currently available which enable an individual to dose a predetermined quantity of insulin such as, for example, a hypodermic syringe, an insulin pen, and an insulin pump. One such insulin pump is the Animas® Ping, a product which is manufactured by Animas Corporation. Another is the Animas® Vibe, also manufactured by Animas Corporation.

People with diabetes should maintain tight control over their lifestyle, so that they are not adversely affected by, for example, irregular food consumption or exercise. In addition, a physician dealing with a particular individual with diabetes may require detailed information on the individual's lifestyle to provide effective treatment or modification of treatment for controlling diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their physician on each visit.

The aforementioned methods of recording lifestyle information are inherently difficult, time consuming, and possibly inaccurate. Paper logbooks are not necessarily always carried by an individual and may not be accurately completed when required. Such paper logbooks are small and it is therefore difficult to enter detailed information requiring detailed descriptors of lifestyle events. Furthermore, an individual may often forget key facts about their lifestyle when questioned by a physician who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system, requires a laborious transcription of information, including lifestyle data, into this secondary data storage. Difficulty of data recordation encourages retrospective entry of pertinent information that results in inaccurate and incomplete records.

### SUMMARY OF THE DISCLOSURE

Applicant has discovered that the use of certain risk index (i.e., Average Daily Risk Range) is further improved if the components underlying this index is also provided that show the impact of hypoglycemica or hyperglycemia driving the risk range for this index.

In one aspect, a system for management of diabetes of a subject is provided. The system includes at least one glucose monitor, at least one biosensor, and a controller. The at least one glucose monitor is configured to measure a glucose concentration based on an enzymatic reaction with physiological fluid in the at least one biosensor that provides an electrical signal representative of the glucose concentration. The controller is in communication with at least one glucose monitor. The controller is configured to receive or transmit glucose levels measured by the glucose monitor over a predetermined time period from the at least one glucose monitor and pump for determination of an average daily risk range with a maximal hyperglycemic value and a maximal hypoglycemic value for each day in the predetermined time period, and in which the maximal hyperglycemic and hypoglycemic values are also annunciated in combination with the daily risk range for each day of the predetermined time period.

In this aspect, the controller is configured to determine the average-daily-risk-range (ADRR) and the maximal hyperglycemic value and maximal hypoglycemic value with the following equations and logical conditions:

$$ADRR = \frac{1}{M} {\sum }_{i = 1}^{M} ( {LR}^{i} + {HR}^{i} )$$

LR=max (RL(BG))
HR=max (RH(BG))
Daily Risk Range for each day is defined as DRR=LR+HR
where ADRR may include the average-daily-risk-range;
i may include the number of days in sequence to M days;
M may include the number of days for which an ADRR value is calculated
LR may include the Maximal Hypoglycemic for each day
HR may include the Maximal Hyperglycemic value for each day
ƒ(BG)=γ([ln(BG)]^{α}−β):
r(BG)=10[ƒ(BG)]²:
Let RL(BG)=R(BG) and if ƒ(BG)<0; else RL(BG)=0
Let RH(BG)=R(BG) if ƒ(BG)>0; else RH(BG)=0
where
   α=1.084 (1.026 if mmol/L);
   β=5.381 (1.861 if mmol/L) and
   γ=1.509 (1.794 if mmol/L).

It is further noted that in this system, the controller is configured to annunciate the maximal hyperglycemic and hypoglycemic values with the daily risk range for each day of the average daily risk range in a visual display. The number of glucose measurements for this system must be at least 3 for each day for the determination of the average daily risk range and the maximal hyperglycemic and hypoglycemic values; and the time period may include any number of days from about one day to about 120 days, or combinations thereof.

In yet another aspect, a method for management of diabetes of a user with at least a glucose monitor, biosensor, and a controller. The method can be achieved by: measuring with the glucose monitor and biosensor a plurality of glucose values in physiological fluid of a user; storing the measured glucose values in a memory of at least one of the monitor and controller; determining an average daily risk range from the glucose values of the storing step for each day of a predetermined time period; calculating a maximal hyperglycemic value and a maximal hypoglycemic value from the stored glucose values for each day of the predetermined time period; and annunciating the average daily risk range and the maximal hyperglycemic and hypoglycemic values for each day of the predetermined time period. In this method, the calculating step may include ascertaining the maximal hyperglycemic and hypoglycemic values for each day with the following equations and logical conditions:
ƒ(BG)=γ([ln(BG)]^{α}−β):
r(BG)=10[ƒ(BG)]²:
Let RL(BG)=R(BG) and if ƒ(BG)<0; else RL(BG)=0
Let RH(BG)=R(BG) if ƒ(BG)>0; else RH(BG)=0
LR=max (RL(BG))
HR=max (RH(BG))
LR may include the Maximal Hypoglycemic for each day
HR may include the Maximal Hyperglycemic value for each day
Daily Risk Range for each day is defined as DRR=LR+HR
where
   α=1.084 (1.026 if mmol/L);
   β=5.381 (1.861 if mmol/L) and
   γ=1.509 (1.794 if mmol/L).

Again, in the method, the determining of the average daily risk range may include calculating the average for each day with an equation of the form:

$$ADRR = \frac{1}{M} {\sum }_{i = 1}^{M} ( {LR}^{i} + {HR}^{i} )$$

where ADRR may include the average-daily-risk-range;
i may include the number of days in sequence to M days;
M is the number of days.

Furthermore, in the method, the annunciating may include displaying the maximal hyperglycemic and hypoglycemic values in one Cartesian graph with one axis representing glucose values and the other axis representing the number of days and displaying the daily risk range for each day of the average daily risk range in another Cartesian graph with one axis representing a risk range from low, medium, high and the other axis representing the number of days. It is noted that a number of glucose measurements must be at least 3 for each day for the determination of the average daily risk range and the maximal hyperglycemic and hypoglycemic values; and the predetermined time period may include any number of days from about one day to about 120 days, or combinations thereof.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 illustrates an exemplary embodiment of the diabetic management system.

FIG. 2 illustrates an exemplary logic diagram of the technique utilized by the system of FIG. 1.

FIG. 3A illustrates the total daily risk range from glucose measurements made in a predetermined time period, such as one day.

FIG. 3B illustrates the components of the daily risk range of the glucose measurements of FIG. 3A.

### MODES FOR CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment. Furthermore, the term “user” includes not only the patient using a drug infusion device but also the caretakers (e.g., parent or guardian, nursing staff or home care employee). The term “drug” may include pharmaceuticals or other chemicals that causes a biological response in the body of a user or patient.

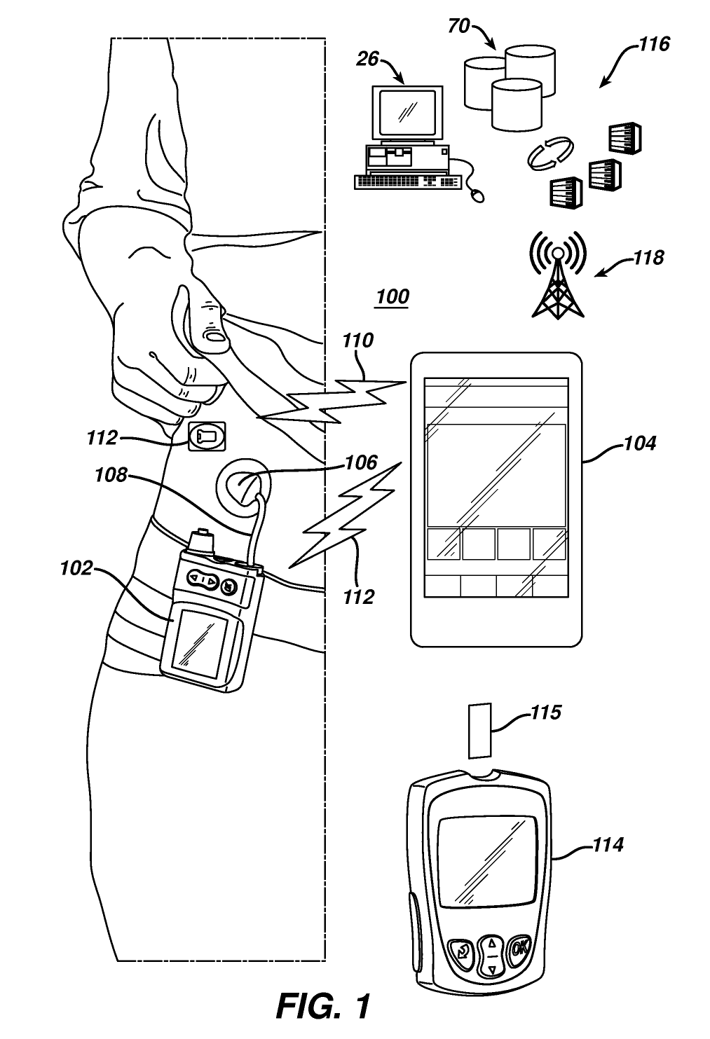

FIG. 1 illustrates a drug delivery system 100 according to an exemplary embodiment. Drug delivery system 100 includes a drug delivery device 102 and a remote controller 104. Drug delivery device 102 is connected to an infusion set 106 via flexible tubing 108.

Drug delivery device 102 is configured to transmit and receive data to and from remote controller 104 by, for example, radio frequency communication 110. Drug delivery device 102 may also function as a stand-alone device with its own built in controller. In one embodiment, drug delivery device 102 is a drug infusion device and remote controller 104 is a hand-held portable controller. In such an embodiment, data transmitted from drug delivery device 102 to remote controller 104 may include information such as, for example, drug delivery data, blood glucose information, basal, bolus, insulin to carbohydrates ratio or insulin sensitivity factor, to name a few. The controller 104 may be configured to receive continuous analyte readings from a continuous analyte (“CGM”) sensor 112. Data transmitted from remote controller 104 to drug delivery device 102 may include analyte test results and a food database to allow the drug delivery device 102 to calculate the amount of drug to be delivered by drug delivery device 102. Alternatively, the remote controller 104 may perform dosing or bolus calculation and send the results of such calculations to the drug delivery device. In an alternative embodiment, an episodic blood analyte meter 114 may be used alone or in conjunction with the CGM sensor 112 to provide data to either or both of the controller 102 and drug delivery device 102. Alternatively, the remote controller 104 may be combined with the meter 114 into either (a) an integrated monolithic device; or (b) two separable devices that are dockable with each other to form an integrated device. Each of the devices 102, 104, and 114 has a suitable micro-controller (not shown for brevity) programmed to carry out various functionalities. For example, a microcontroller can be in the form of a mixed signal microprocessor (MSP) for each of the devices 102, 104, or 114. Such MSP may be, for example, the Texas Instrument MSP 430, as described in patent application publication numbers US2010-0332445, and US2008-0312512 which are incorporated by reference in their entirety herein and attached hereto the Appendix of this application. The MSP 430 or the pre-existing microprocessor of each of these devices can be configured to also perform the method described and illustrated herein.

The measurement of glucose can be based on a physical transformation (i.e., the selective oxidation) of glucose by the enzyme glucose oxidase (GO). For example, in the strip type biosensor, the reactions that can occur in such biosensor are summarized below in Equations 1 and 2.

Glucose+GO₍ₒₓ₎→Gluconic Acid+GO_{(red)} Eq. 1

GO_{(red)}+2Fe(CN)₆³⁻→GO₍ₒₓ₎+2Fe(CN)₆⁴⁻ Eq. 2

As illustrated in Equation 1, glucose is oxidized to gluconic acid by the oxidized form of glucose oxidase (GO₍ₒₓ₎). It should be noted that GO₍ₒₓ₎ may also be referred to as an “oxidized enzyme.” During the chemical reaction in Equation 1, the oxidized enzyme GO₍ₒₓ₎ is transformed to its reduced state, which is denoted as GO_{(red)} (i.e., “reduced enzyme”). Next, the reduced enzyme GO_{(red)} is re-oxidized back to GO₍ₒₓ₎ by reaction with Fe(CN)₆³⁻ (referred to as either the oxidized mediator or ferricyanide) as illustrated in Equation 2. During the re-generation or transformation of GO_{(red)} back to its oxidized state GO₍ₒₓ₎, Fe(CN)₆³⁻ is reduced to Fe(CN)₆⁴⁻ (referred to as either reduced mediator or ferrocyanide).

When the reactions set forth above are conducted with a test voltage applied between two electrodes, a test current can be created by the electrochemical re-oxidation of the reduced mediator at the electrode surface. Thus, since, in an ideal environment, the amount of ferrocyanide created during the chemical reaction described above is directly proportional to the amount of glucose in the sample positioned between the electrodes, the test current generated would be proportional to the glucose content of the sample. A mediator, such as ferricyanide, is a compound that accepts electrons from an enzyme such as glucose oxidase and then donates the electrons to an electrode. As the concentration of glucose in the sample increases, the amount of reduced mediator formed also increases; hence, there is a direct relationship between the test current, resulting from the re-oxidation of reduced mediator, and glucose concentration. In particular, the transfer of electrons across the electrical interface results in the flow of a test current (2 moles of electrons for every mole of glucose that is oxidized). The test current resulting from the introduction of glucose can, therefore, be referred to as a glucose current.

Analyte levels or concentrations can also be determined by the use of the CGM sensor 112. The CGM sensor 112 utilizes amperometric electrochemical sensor technology to measure analyte with three electrodes operably connected to the sensor electronics and are covered by a sensing membrane and a biointerface membrane, which are attached by a clip. The top ends of the electrodes are in contact with an electrolyte phase (not shown), which may include a free-flowing fluid phase disposed between the sensing membrane and the electrodes. The sensing membrane may include an enzyme, e.g., analyte oxidase, which covers the electrolyte phase. In this exemplary sensor, the counter electrode is provided to balance the current generated by the species being measured at the working electrode. In the case of an analyte oxidase based glucose sensor, the species being measured at the working electrode is H₂O₂. The current that is produced at the working electrode (and flows through the circuitry to the counter electrode) is proportional to the diffusional flux of H₂O₂. Accordingly, a raw signal may be produced that is representative of the concentration of blood glucose in the user's body, and therefore may be utilized to estimate a meaningful blood glucose value. Details of the sensor and associated components are shown and described in U.S. Pat. No. 7,276,029, which is incorporated by reference herein as if fully set forth herein this application. In one embodiment, a continuous analyte sensor from the Dexcom Seven System (manufactured by Dexcom Inc.) can also be utilized with the exemplary embodiments described herein.

Drug delivery device 102 may also be configured for bi-directional wireless communication with a remote health monitoring station 116 through, for example, a wireless communication network 118. Remote controller 104 and remote monitoring station 116 may be configured for bi-directional wired communication through, for example, a telephone land based communication network. Remote monitoring station 116 may be used, for example, to download upgraded software to drug delivery device 102 and to process information from drug delivery device 102. Examples of remote monitoring station 116 may include, but are not limited to, a personal or networked computer, a personal digital assistant, other mobile telephone, a hospital base monitoring station or a dedicated remote clinical monitoring station.

Drug delivery device 102 includes processing electronics including a central processing unit and memory elements for storing control programs and operation data, a radio frequency module 116 for sending and receiving communication signals (i.e., messages) to/from remote controller 104, a display for providing operational information to the user, a plurality of navigational buttons for the user to input information, a battery for providing power to the system, an alarm (e.g., visual, auditory or tactile) for providing feedback to the user, a vibrator for providing feedback to the user, a drug delivery mechanism (e.g. a drug pump and drive mechanism) for forcing a drug from a drug reservoir (e.g., a drug cartridge) through a side port connected to an infusion set 106 and into the body of the user.

The components of the system described in relation to FIG. 1 are helpful to the person with diabetes in managing their disease. However, to achieve the efficicacy in management of the disease, the person with diabetes would need more than just these components. As applicant has recognized, the component or the system must be able to provide easy to understand information that assist in the decision making of the person. To assist in this, an index called Average Daily Risk Range (ADRR) Index was invented at the University of Virginia by Boris Kovatchev (http://care.diabetesjournals.org/content/29/11/2433.full.pdf) with a copy attached to the Appendix of this application, which reference is incorporated by reference herein into this application. Details of the derivation for the ADRR is provided by U.S. Patent/Publication Number: US20090171589A1 Publication Date: Jul. 2, 2009, title: METHOD, SYSTEM AND COMPUTER PROGRAM PRODUCT FOR EVALUATION OF BLOOD GLUCOSE VARIABILITY IN DIABETES FROM SELF-MONITORING DATA; Inventor: Kovatchev, Boris P., and incorporated by reference as if fully set forth herein. The ADRR Index is designed to provide a “risk index” for a patient with diabetes that explains the overall risk they have for adverse events due to glucose control. For example, a patient might be provided with an ADRR Index of “23” in their daily report on their meter, pump, or controller. While this number is associated with medium risk, it is not clear how this number relates to the patient's high and low glucose concentration (when both may contribute to the risk) and when the patient may be able to improve their blood glucose during a week involving days with both high and low values despite the medium risk index.

While the ADRR Index provides a simple number and category, it can be difficult for doctors and patients to understand the statistic and what contributes to its value. This invention transforms the input components of ADRR to provide a better understanding of the internals of the ADRR Index and how it is affected by the patient's blood glucose (“BG”). At this point, it is worthwhile to discuss how the ADRR Index is determined. In particular, the glucose risk function defines a way of noting the risk of each reading R(BG) for each day. In one example, a daily risk range is determined as follows:

ƒ(BG)=γ([ln(BG)]^{α}−β): Eq. 3.

Equation 3 is scale function f of a blood glucose reading value is provided to convert an interval ranging from 20 to 600 into an interval of −\sqrt{10} to \sqrt{10}, with a zero at 112.5.

r(BG)=10[ƒ(BG)]²: Eq. 4

Equation 4 is the risk value associated with a blood glucose reading. If ƒ(BG)<0, then RLᵢ=r(BG), else RLᵢ=0 This relationship indicates the low risk value associated with i^{th} blood glucose reading, where 1≦i≦N. That is, if the function ƒ is less than zero then RLᵢ is set to equal to Eq. 4, otherwise, RLᵢ is set to equal to zero. On the other hand, if ƒ(BG)≧0, then RHᵢ=r(BG), else RHᵢ=0 This relationship is indicative of the high risk value associated with blood glucose reading, where 1≦i≦N. That is, if the function ƒ is equal to or greater than zero then RHᵢ is set to approximate equal to Equation 4 otherwise RHᵢ is set to equal to zero.

A maximal value of the hypoglycemic values on a certain day is defined as Maxⱼ (RLᵢ): which is the maximum RLᵢ value among all i^{th} readings that fall on day Dⱼ. On the other hand, a maximal value of the hyperglycemic values on a certain day is defined as Maxⱼ (RFᵢ): which is the maximum RHᵢ value among all i^{th} readings that fall on day Dⱼ. If the reading had a positive f(BG) value then the risk is from high blood glucose RH and if the reading had a negative f(BG) value, then the risk is from low blood glucose RL. Consequently, ADRR defines the daily risk range as the sum of Max(RH) and Max(RL) in each day where at least 3 blood glucose readings are present.

To determine an average of such daily risk range over an interval of predetermined time (e.g, M number of days), Equations 3 and 4 are utilized where α=1.084 (1.026 if mmol/L); β=5.381 (1.861 if mmol/L) and γ=1.509 (1.794 if mmol/L). Then, the following operations can be made:

Let R(BG)=10×ƒ(BG)² Eq. 5

Let RL(BG)=R(BG) and if ƒ(BG)<0; else RL(BG)=0 Eq. 6

Let RH(BG)=R(BG) if ƒ(BG)>0; else RH(BG)=0 Eq. 7

where the maximal hypoglycemic value LR=Max(RL(BG)) for each day Eq. 8

where the maximal hyperglycemic value HR=Max(RH(BG)) for each day Eq. 9

Daily Risk Range for each day is defined as DRRⁱ=LRⁱ+HRⁱ Eq. 10

$$From ADRR = \frac{1}{M} {\sum }_{i = 1}^{M} ( {LR}^{i} + {HR}^{i} ) \qquad Eq . 11$$

where M is the number of days for which a DRR value is calculated
i.e., days where ≧3 BG values are present.

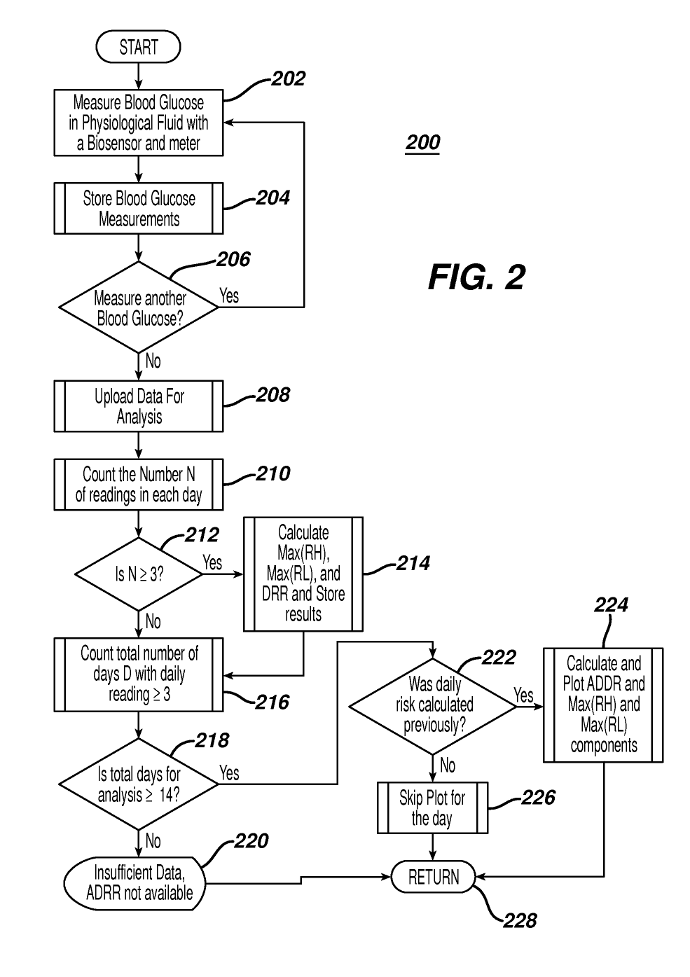

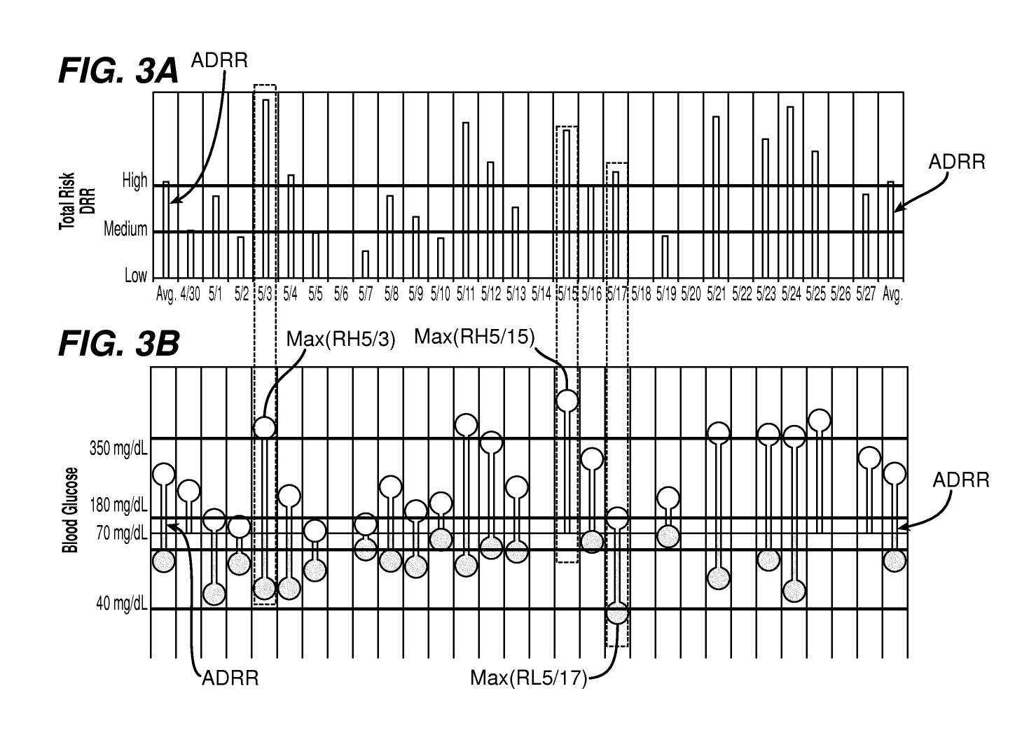

Referring to FIG. 2, a logic diagram of the technique 200 utilized by applicant is illustrated. In step 202, a blood glucose measurement is made by a patient using the glucose monitor and a biosensor (e.g., SMBG or CGM). The measurement is made via a physical transformation of glucose in the physiological sample into an enzymatic product, and the measurement is stored at step 204. The patient may measure his or her glucose a short time thereafter in step 206, at which time the logic reverts to step 202. Assuming that the patient has measured glucose several times a day over several days, the data can be utilized for analysis or uploaded into a server for analysis at step 208. At step 210, the logic looks for a number “N” of blood glucose measurements each day. If N is greater than or equal to 3, (i.e., at least 3 measurements a day), then the logic moves from step 212 to 214 at which a calculation of the maximal of the risk from high glucose measurements (i.e., Max(RH)) or the maximal of the risk from low glucose measurements (i.e., Max(RL)) and the total risk, in the form a-daily-risk-range (i.e., DRR) from the glucose measurements are made for each day. At step 216, the logic determines the number of days “D” with daily measurements of at least 3 glucose measurements. The logic determines at step 218 whether the total number of D days is at least 14 days. If false then the logic returns a message at step 220 that insufficient data have been provided for determination of ADRR. If true at step 218, the logic queries whether the daily risk range DRR was calculated previously. If true then the logic plots FIGS. 3A and 3B to annunciate at least one of ADRR, DRR, Max(RH) and Max(RL) otherwise if false at step 222, the annunciation of the risk factors is skipped for that day. The logic returns to step 228 to the main routine thereafter step 224 or 226. As used here, the term “annunciate” or “annunciating” and variations on the root term indicate that an announcement may be provided via text, audio, visual or a combination of all modes of communication on the analyte sensor, drug infusion device, or a remote communication device such as a mobile phone, network server, or remote monitoring system for a user, caretaker (e.g., parents, guardian, nursing staff and the like) or a health care provider.

Referring to FIG. 3A, an annunciation of risk factors (in the form of average daily risk range (“ADRR”)) is shown for each day. In FIG. 3B, a corresponding illustration of the maximal hyperglycemic values RH1 . . . RHn and maximal hypoglycemic value RL1, RL2, RL3 . . . RLn for each day of n days are shown. The Max(RH) value for each day is plotted as a positive value, and is noted as the red bars extending above the line. The Max(RL) value for each day is plotted as a negative value, and is noted as the blue bars extending below the line. The maximal hyperglycemic and hypoglycemic values Max(RH) and Max(RL) are used to assist the person with diabetes with the insight as to where the person could improve on control of the blood glucose without particular focusing on any one glucose measurement.

To provide convenient markers of the Max(RH) and Max(RL) values described above, icons or symbols such as, for example, a colored circle of a suitable color or combinations of color and icons can be utilized. The center of Max(RH) could be designated as one colored circle (or polygon) and the center of Max(RL) can be designated as another colored circle (or polygon). Both circles have a fixed radius, the fixed radius can serve as an additional marker the low and high components of the risk. An alternate technique would be to still center the circles on the Max(RH) and Max(RL) values, but to size them according to the value of Max(RH) and Max(RL).

In this alternate solution the area of the circle could be configured to change linearly with the risk. A minimum circle radius, which would correspond to the circle to draw with a risk of 0, is defined and a maximum circle radius, which would correspond to the circle to draw with a risk of 100. The radius of the circle can be calculated using: radius=SQRT ((MaxRadius²−MinRadius²)*(risk/100)+MinRadius²). This would ensure that the areas of the circles drawn would vary correctly with the risk in each day.

Referring back to FIG. 3A, the ADRR for this particular patient is indicated at by the indicators bracketing the range DRR indices (indicated here with the nomenclature “ADRR” and respective lead lines in FIG. 3A) which means that throughout the reporting period from April 30 to May 27, the patient shows an “average” daily risk range that is considered high. The daily risk range DRR is shown in each of the days from April 30 to May 27. While the average or daily risk range DRR gives the patient a good idea that his or her glycemic control may not be optimal, it may not provide the patient with more useful indicators of the components that go into increasing the daily risks. For example, low glucose values are believed to be riskier than high glucose values and that days with both low and high glucose value are believed to be riskier than days with only low or high glucose values.

By providing an insight into the components (maximal hypoglycemia and maximal hyperglycemia) that drive the risk range (e.g., ADRR or DRR) in the form of FIG. 3B along with the ADRR and DRR, applicant is able to provide the patient with a deeper insight into risk areas, i.e., whether it is the high glucose values or the low glucose values that are causing the ADRR or DRR to rise or stay high. Several examples will be discussed in relation to FIGS. 3A and 3B to show the advantages of applicant's invention.

In FIG. 3A, it can be seen, for example, that the DRR for May 3 is indicative of very high risk. However, the patient is not able to discern whether this high risk is caused by very high blood glucose, very low blood glucose or both high and low blood glucose values. By turning to applicant's invention (as embodied in FIG. 3B), it is clear that on this day the maximal of hyperglycemia Max(RH5/3) is high along with the maximal of the hypoglycemia Max(RL5/3) is low, thereby both contributing the high risk indicative in the DRR of May 3.

In another example, indicated on FIG. 3A as May 15, the DRR for this day is also very high but without applicant's invention, the patient would not be able to discern what components of high or low blood glucose values are contributing to the high risk shown in FIG. 3A. However, with the annunciation of FIG. 3B, it can be seen that virtually all of the risks came from the maximal hyperglycemia Max(RH5/15). Maximal value Max(RH5/15) indicates that on this day, virtually all of the risks came from high blood glucose measured on May 15.

On the other hand, on May 17, the patient's DRR in FIG. 3A is showing a high level of risk that, without FIG. 3B, would not provide the patient the required insight into which components of high or low glucose values are contributing to this risk. By turning to FIG. 3B, it can be seen that the majority of the risk came from low glucose values measured during May 17.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A system for management of diabetes of a subject, the system comprising:
at least one glucose monitor that is configured to measure a glucose concentration based on an enzymatic reaction with physiological fluid in a biosensor that provides an electrical signal representative of the glucose concentration; and
a controller in communication with at least one glucose monitor, the controller being configured to receive or transmit glucose levels measured by the glucose monitor over a predetermined time period from the at least one glucose monitor and pump for determination of an average daily risk range with a maximal hyperglycemic value and a maximal hypoglycemic value for each day in the predetermined time period; and
wherein the maximal hyperglycemic and hypoglycemic values are also annunciated in combination with the daily risk range for each day of the predetermined time period.

2. The system of claim 1, in which the controller is configured to determine the average-daily-risk-range (ADRR) and the maximal hyperglycemic value and maximal hypoglycemic value with the following equations and logical conditions:
$$ADRR = \frac{1}{M} {\sum }_{i = 1}^{M} ( {LR}^{i} + {HR}^{i} )$$
LR=max (RL(BG))
HR=max (RH(BG))
Daily Risk Range for each day is defined as DRR=LR+HR
where ADRR comprises the average-daily-risk-range;
i comprises the number of days in sequence to M days;
M comprises the number of days for which a ADRR value is calculated
LR comprises the Maximal Hypoglycemic for each day
HR comprises the Maximal Hyperglycemic value for each day
ƒ(BG)=γ([ln(BG)]^{α-β):}
r(BG)=10[ƒ(BG)]²:
Let RL(BG)=R(BG) and if ƒ(BG)<0; else RL(BG)=0
Let RH(BG)=R(BG) if ƒ(BG)>0; else RH(BG)=0
where
α=1.084 (1.026 if mmol/L);
β=5.381 (1.861 if mmol/L) and
γ=1.509 (1.794 if mmol/L).

3. The system of claim 2, in which the controller is configured to annunciate the maximal hyperglycemic and hypoglycemic values are also annunciated in combination with the daily risk range for each day of the average daily risk range in a visual display.

4. The system of claim 3, in which a number of glucose measurements must be at least 3 for each day for the determination of the average daily risk range and the maximal hyperglycemic and hypoglycemic values.

5. The system of claim 4, in which the time period comprises any number of days from about one day to about 120 days, or combinations thereof.

6. A method for management of diabetes of a user with at least a glucose monitor, biosensor, and a controller, the method comprising the steps of:
measuring with the glucose monitor and biosensor a plurality of glucose values in physiological fluid of a user;
storing the measured glucose values in a memory of at least one of the monitor and controller;
determining an average daily risk range from the glucose values of the storing step for each day of a predetermined time period;
calculating a maximal hyperglycemic value and a maximal hypoglycemic value from the stored glucose values for each day of the predetermined time period; and
annunciating the average daily risk range and the maximal hyperglycemic and hypoglycemic values for each day of the predetermined time period.

7. The method of claim 6, in which the calculating step comprises ascertaining the maximal hyperglycemic and hypoglycemic values for each day with the following equations and logical conditions:
ƒ(BG)=γ([ln(BG)]^{α}−β):
r(BG)=10[ƒ(BG)]²:
Let RL(BG)=R(BG) and if ƒ(BG)<0; else RL(BG)=0
Let RH(BG)=R(BG) if ƒ(BG)>0; else RH(BG)=0
LR=max (RL(BG))
HR=max (RH(BG))
LR comprises the Maximal Hypoglycemic for each day
HR comprises the Maximal Hyperglycemic value for each day
Daily Risk Range for each day is defined as DRR=LR+HR
where
α=1.084 (1.026 if mmol/L);
β=5.381 (1.861 if mmol/L) and
γ=1.509 (1.794 if mmol/L).

8. The method of claim 7, in which the determining of the average daily risk range comprises calculating the average for each day with an equation of the form:
$$ADRR = \frac{1}{M} {\sum }_{i = 1}^{M} ( {LR}^{i} + {HR}^{i} )$$
where ADRR comprises the average-daily-risk-range;
i comprises the number of days in sequence to M days;
M is the number of days.

9. The method of claim 8, in which the annunciating comprises displaying the maximal hyperglycemic and hypoglycemic values in one Cartesian graph with one axis representing glucose values and the other axis representing the number of days and displaying the daily risk range for each day of the average daily risk range in another Cartesian graph with one axis representing a risk range from low, medium, high and the other axis representing the number of days.

10. The method of claim 3, in which a number of glucose measurements must be at least 3 for each day for the determination of the average daily risk range and the maximal hyperglycemic and hypoglycemic values.

11. The method of claim 8, in which the predetermined time period comprises any number of days from about one day to about 120 days, or combinations thereof.
